(19) United States
(12) Patent Application Publication
(10) Pub. No.: US 2021/0189262 A1
(43) Pub. Date: Jun. 24, 2021

(54) METHOD FOR SELECTIVE REMOVAL OF POLYCYCLIC AROMATIC HYDROCARBONS FROM OILS OBTAINED AS A RESULT OF PETROLEUM PROCESSING

(71) Applicants: POLYMEMTECH SP. Z O.O., Warszawa (PL); GRUPA LOTOS S.A., Gdansk (PL)

(72) Inventors: Andrzej KOWALSKI, Warszawa (PL); Marek ROGUSKI, Warszawa (PL); Wojciech PIATKIEWICZ, Warszawa (PL); Maciej SZWAST, Warszawa (PL); Tomasz WOLYNKO, Warszawa (PL); Dagmara APTOWICZ, Gdansk (PL); Jan BIEDRON, Gdansk (PL); Kazimierz KOWALCZYK, Gdansk (PL); Michal SZARO, Gdansk (PL)

(21) Appl. No.: 16/080,463

(22) PCT Filed: Feb. 28, 2017
(86) PCT No.: PCT/PL2017/050011

§ 371 Date: Aug. 28, 2018

(30) Foreign Application Priority Data
Feb. 29, 2016 (PL) P.416295

(51) Int. Cl. C10G 53/08; B01J 20/20; B01J 20/28; B01D 15/08; B01D 61/14; B01D 61/16; B01D 69/02

(52) U.S. Cl. CPC C10G 53/08; B01D 2325/02; B01J 20/28064; B01J 20/28066; B01J 20/28085; B01D 15/08; B01D 61/147; B01D 61/16; B01D 69/02; C10G 2300/308; C10G 2300/302; C10G 2300/202; B01D 2311/04; B01D 2311/2626; B01J 20/20

(57) ABSTRACT

A method for selective removal of polycyclic aromatic hydrocarbons from oils obtained as a result of petroleum processing, including two separate processes: filtration through a porous carbon-containing bed comprising and filtration through microfiltration membranes. The method is particularly useful for purifying oils selected from unconverted oils obtained in hydrocracking processes, products of further processing of these oils, engine oil and used engine oil.

### FIELD

The aspects of the disclosed embodiments relate to a method for selective removal of polycyclic aromatic hydrocarbons (PAHs) from oils obtained as a result of petroleum processing, in particular from unconverted oils obtained in hydrocracking processes, products of further processing of these oils, engine oil and used engine oil.

### PRIOR ART

### Problem

Oils obtained as a result of petroleum processing, including unconverted oils obtained in hydrocracking processes and products obtained therefrom contain polycyclic aromatic hydrocarbons such as pyrene, benzo(a)pyrene, dibenzo(a, g, h)pyrene, dibenzo(a, h)anthracene, chrysene, coronene and others, which include in their structure three or more condensed aromatic rings.

Polycyclic aromatic hydrocarbons (PAHs) accelerate catalyst deactivation in refinery and petrochemical catalytic processes. PAHs reduce also the effective utility of the products obtained.

Irradiated with visible light in the presence of oxygen PAHs undergo a photochemical reaction resulting in formation of undesired chemical compounds, i.a. diols, quinones and aldehydes. These compounds also tend to precipitate in form of sediments. Additionally the polycyclic aromatic hydrocarbons show carcinogenic properties, and pose a threat to human health and the environment. The proposed method based on an integrated filtration process enables to solve all these problems.

### Worldwide Level (Literature)

1) M. B. Gawlik, Maciej Bilek, “Możliwość obniźenia emisji wielopierścieniowych węglowodorów aromatycznych ze źródel antropogennych” [“The possibilities of decrease of emission of polycyclic aromatic hydrocarbons from anthropogenic sources”], katedra Toksykologii C M Uniwersytet Jagielloński, Medycyna Środowiska 2006.
2) Zsolt Kemény, Gabriella Hellner, Andrea Radnóti, Timo Erjomaa, Polycyclic Aromatic Hydrocarbon Removal from Coconut Oil, Euro Fed Lipid meeting, Rotterdam 2011
3) Method of removing contaminants from petroleum distillates, U.S. Pat. No. 6,320,090 B1
4) Selective multi-ring aromatics extraction using a porous, non-selective partition membrane barrier, U.S. Pat. No. 5,045,206 A
5) Neha Budhwani, Removal of Polycyclic Aromatic Hydrocarbons Present in Tyre Pyrolytic Oil Using Low Cost Natural Adsorbents, International Journal of Biological, Biomolecular, Agricultural, Food and Biotechnological Engineering Vol:9, No:2, 2015
6) Gong Z., Alef K., Wilke B. M., Li P., Activated carbon adsorption of PAHs from vegetable oil used in soil remediation, J Hazard Mater. 2007 May 8;143(1-2):372-8

7) D. González , L. M. Ruiz , G. Garralón , F. Plaza , J. Arévalo, J. Parada, J. Pérez, B. Moreno, Migual Ángel Gómez, Wastewater polycyclic aromatic hydrocarbons removal by membrane bioreactor, Desalination and Water Treatment, 42 (2012) 94-99

### SUMMARY

The separation (purification) method is based on two-step process:
Filtration process carried out on carbon-containing bed for selective adhesion to its surface of undesired polycyclic aromatic hydrocarbons (PAHs) from oils obtained as a result of petroleum processing, including unconverted oils obtained in hydrocracking processes and products of further processing of these oils.
Filtration process for removing bed particles containing adhered PAHs from oils obtained as a result of petroleum processing, including unconverted oils obtained in hydrocracking processes and products of further processing of these oils.

Preferably, the filtration is carried out on the carbon-containing bed in granulated or powdered form having extended surface of 500-1600 m²/g.

Preferably, the filtration is carried out on the carbon-containing bed having grain size of 0.3-4 mm.

Preferably, the filtration process temperature on the carbon-containing bed ranges from 10 to 90° C., in particular from 17 to 65° C.

Preferably, the filtration is carried out on the carbon-containing bed at linear velocity ranging from 1 to 10 m/min.

Preferably, the filtration is carried out on the microfiltration membranes with the nominal pore size ranging from 0.1 to 1.2 micrometers, and in particular from 0.1 to 0.5 micrometers.

The above-mentioned process parameters do not affect the mutual separation of desired hydrocarbons contained in the oils obtained as a result of petroleum processing, including the unconverted oils obtained in hydrocracking processes and products of further processing of these oils, but only result in selective removal of polycyclic aromatic hydrocarbons.

### BRIEF DESCRIPTION OF DRAWINGS

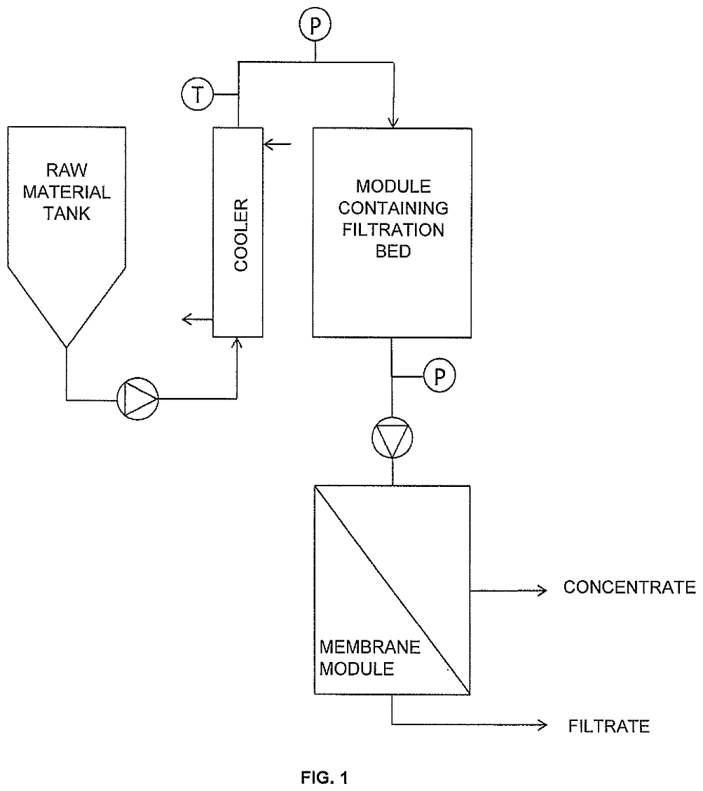

The present disclosure in exemplary embodiment was illustrated in a drawing. FIGURE 1 shows a schematic diagram of realisation of the method according to the aspects of the disclosed embodiments.

### EXPERIMENTS

Following tests were carried out, which according to the authors show high efficiency in solving the problem.

Oil sample used in all the tests had the physicochemical properties as shown in the following table.

| Appearance at temp. 20° C. | clear, straw-yellow colour |
|---|---|
| Appearance at temp. 20° C. | no suspensions |
| Density g/cm³ 15° C. | 0.8456 |
| Absorbance at wavelength of 385 nm | 1.0925 |
| in isooctane solution | |
| Kinematic viscosity at 100° C. cSt | 5.24 |
| Kinematic viscosity at 40° C. cSt | 27.9 |
| Viscosity index | 121 |
| Sulphur content %(m/m) | 0.006 |

### Test 1

An oil sample was subjected to an in-depth oxidation by means of UV radiation and titanium dioxide as a catalyst. Irradiation time was 30 minutes.

Resultant sample was filtered in a cross-flow filtration system using a system of single-stage filtration on microporous membranes.

### Test 2

An oil sample was subjected to an in-depth oxidation by means of UV radiation and titanium dioxide as a catalyst. Irradiation time was 42 minutes.

Resultant sample was filtered in a cross-flow filtration system using microfiltration membranes.

Further the sample was filtered through a four-stage integrated filtration system.

Resultant sample was filtered in a cross-flow filtration system using nanofiltration membranes.

### Test 3

An oil sample was filtered through a three-stage integrated filtration system using carbon-containing bed and filtration on filtration membranes.

### Test 4

An oil sample was filtered through a two-stage integrated filtration system using carbon-containing bed and filtration on filtration membranes.

### Test Results

Test 1:

| Appearance at temp. 20° C. | clear, dark straw-yellow colour |
|---|---|
| Appearance at temp. 20° C. | no suspensions |
| Density g/cm³ 15° C. | 0.8456 |
| Absorbance at wavelength of 385 nm | 0.9882 |
| in isooctane solution | |
| Kinematic viscosity at 100° C. cSt | 5.236 |
| Kinematic viscosity at 40° C. cSt | 27.92 |
| Sulphur content %(m/m) | 0.0063 |

Test 2:

| Appearance at temp. 20° C. | clear, dark straw-yellow colour |
|---|---|
| Appearance at temp. 20° C. | no suspensions |
| Density g/cm³ 15° C. | 0.8456 |
| Absorbance at wavelength of 385 nm | 0.0466 |
| in isooctane solution | |
| Kinematic viscosity at 100° C. cSt | 5.804 |
| Kinematic viscosity at 40° C. cSt | 32.63 |
| Sulphur content %(m/m) | 0.004 |

Test 3:

| Appearance at temp. 20° C. | clear, dark straw-yellow colour |
|---|---|
| Appearance at temp. 20° C. | no suspensions |
| Density g/cm³ 15° C. | 0.8456 |
| Absorbance at wavelength of 385 nm | 0.0970 |
| in isooctane solution | |
| Kinematic viscosity at 100° C. cSt | 5.811 |
| Kinematic viscosity at 40° C. cSt | 32.43 |
| Sulphur content %(m/m) | 0.0044 |

Test 4:

| Appearance at temp. 20° C. | clear, dark straw-yellow colour |
|---|---|
| Appearance at temp. 20° C. | no suspensions |
| Density g/cm³ 15° C. | 0.8456 |
| Absorbance at wavelength of 385 nm | 0.5512 |
| in isooctane solution | |
| Kinematic viscosity at 100° C. cSt | 5.513 |
| Kinematic viscosity at 40° C. cSt | 29.8 |
| Sulphur content %(m/m) | 0.0052 |

### Discussion of the Results

Basic parameter defining the PAH separation degree was UV absorbance of isooctane solutions of the same concentration at different wavelengths. In the tables above absorbance results were provided for a single wavelength.

Absorbance value at wavelength of 385 nm in isooctane solution being lower than 0.1500 can be considered a satisfactory result.

### Test 1

Absorbance at wavelength of 385 nm in isooctane solution changed slightly (the change was within the margin of error)

The colour of resultant filtrate was much darker than the starting oil sample.

### Test 2

The absorbance value obtained at wavelength of 385 nm in isooctane solution amounting to 0.0466 can be considered very good.

### Test 3

The result of test 3 is satisfactory, absorbance at wavelength of 385 nm in isooctane solution changed significantly and amounted to 0.0970.

### Test 4

The result of test 4 may be considered unsatisfactory. The sample obtained in test 4 the absorbance at wavelength of 385 nm in isooctane solution was reduced merely to 0.5512.

### Conclusions

Polycyclic aromatic hydrocarbons (PAHs) have molar weights similar to saturated hydrocarbons constituting the components of oils obtained as a result of petroleum processing, including unconverted oils obtained in hydrocracking processes and products of further processing of these oils. Separating PAHs from saturated hydrocarbons by means of filtration membranes only did not give expected separation results.

The most preferred is the method used in test 3.

1. A method for selective removal of polycyclic aromatic hydrocarbons from oils obtained as a result of petroleum processing, characterised in that it comprises two separate processes: filtration through a porous carbon-containing bed comprising and filtration through microfiltration membranes.

2. The method according to claim 1, characterised in that the oils obtained as a result of petroleum processing are selected from: unconverted oils obtained in hydrocracking processes, products of further processing of these oils, engine oil and used engine oil.

3. The method according to claim 1, wherein the filtration is carried out on the carbon-containing bed in granulated or powdered form having extended surface of 500-1600 m²/g.

4. The method according to claim 1, wherein the filtration is carried out on the carbon-containing bed having grain size of 0.3-4 mm.

5. The method according to claim 1, wherein the filtration process temperature on the carbon-containing bed ranges from 10 to 90° C.

6. The method according to claim 5, wherein the filtration process temperature on the carbon-containing bed ranges from 17 to 65° C.

7. The method according to claim 1, wherein the filtration is carried out on the carbon-containing bed at linear velocity ranging from 1 to 10 m/min.

8. The method according to claim 1, wherein the filtration is carried out on the microfiltration membranes with the nominal pore size ranging from 0.1 to 1.2 micrometers.

9. The method according to claim 1, wherein the filtration is canned out on the microfiltration membranes with the nominal pore size ranging from 0.1 to 0.5 micrometers.
